(19) United States
(12) Plant Patent Application Publication
(10) Pub. No.: US 2015/0334900 P1
(43) Pub. Date: Nov. 19, 2015

(54) Dahlia plant named 'KLEDH13033'

(50) ;

(71) Applicant: Klemm+Sohn GmbH & Co. KG, Stuttgart (DE)

(72) Inventors: Nils Klemm, Stuttgart (DE); Silke Wagener, Leinfelden-Echterdingen (DE)

(73) Assignee: Klemm+Sohn GmbH & Co. KG, Stuttgart (DE)

(21) Appl. No.: 14/120,257

(22) Filed: May 13, 2014

(51) Int. Cl. A01H 5/00

(52) U.S. Cl. PLT/321

(57) ABSTRACT

A new Dahlia plant particularly distinguished by having yellow ray florets with a red eye and compact, semi-upright, freely basal branching inflorescences held above the foliage, is disclosed.

### GENUS AND SPECIES

Dahlia sp.

### VARIETY DENOMINATION

‘KLEDH13033’

### BACKGROUND OF THE NEW PLANT

The present invention comprises a new and distinct variety of Dahlia, botanically known as Dahlia sp. and hereinafter referred to by the variety name ‘KLEDH13033’. ‘KLEDH13033’ is a selection that results from an open pollination of unknown parents in the summer of 2008. Seed were collected from the open pollination and planted, with a single plant named ‘KLEDH13033’ discovered in Stuttgart, Germany in May of 2009. Initial unrooted cuttings of ‘KLEDH13033’ were taken in May of 2009. ‘KLEDH13033’ was found to reproduce true to type in successive generations of asexual propagation via unrooted cuttings in Stuttgart, Germany.

### SUMMARY

The following are the most outstanding and distinguishing characteristics of this new variety when grown under normal horticultural practices in Stuttgart, Germany.

1. Yellow ray florets with a red eye; and

2. Compact, semi-upright, freely basal branching inflorescences held above the foliage.

### CROSS REFERENCE TO RELATED APPLICATIONS

Plant Breeder's Rights for ‘KLEDH13033’ were applied for in Canada on Jan. 9, 2014, application number 14-8172. ‘KLEDH13033’ has not been made publicly available or sold more than one year prior to the filing date of this application.

### DESCRIPTION OF THE PHOTOGRAPHS

This new Dahlia plant is illustrated by the accompanying photographs which show an overall view of the plant. The colors shown are as true as can be reasonably obtained by conventional photographic procedures.

FIG. 1 shows the overall plant habit of the plant grown in a basket. The plant is about 5 months-old grown in Stuttgart, Germany in May 2013.

FIG. 2 shows the overall plant habit of the plant grown in a pot. The plant is about 3 months-old grown in Stuttgart, Germany in July 2013.

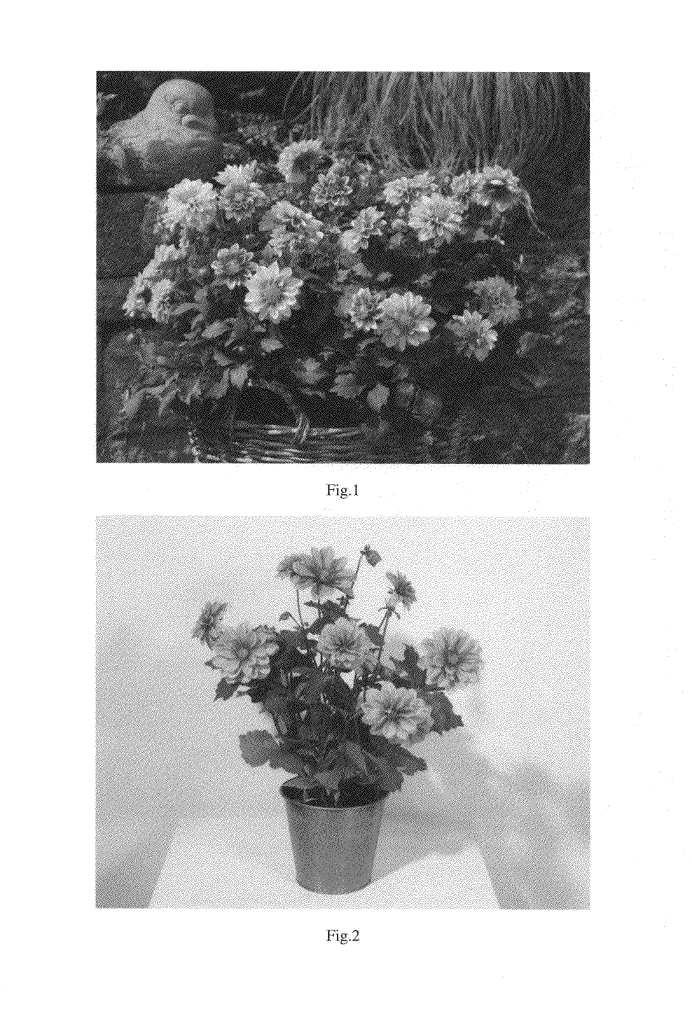

### DESCRIPTION OF THE NEW VARIETY

The following detailed descriptions set forth the distinctive characteristics of ‘KLEDH13033’. The data which define these characteristics were collected from asexual reproductions carried out in Stuttgart, Germany. Data was collected on four month-old plants grown indoors, under natural and artificial light in Stuttgart, Germany in January of 2014. Plants were pinched one time with a growth regulator. Color references are to The R.H.S. Colour Chart of The Royal Horticultural Society of London (R.H.S.), 5^{th} edition (2007).
Classification:
   Family.—Asteraceae.
   Species.—Dahlia sp.
   Common name.—Dahlia.
   Variety.—‘KLEDH13033’.
Parentage: Unknown
Propagation:
   Type.—Asexual, unrooted cuttings.
   Time to produce a rooted young plant.—3 to 4 weeks.
   Root description.—Medium thickness, light tan in colour.
   Rooting habit.—Freely branching, moderately dense.
Plant description:
   Plant and growth habit.—Compact, semi-upright, freely basal branching inflorescences held above the foliar plant.
   Plant height.—25.0 cm.
   Plant diameter.—35.0 cm.
Lateral branches:
   Length.—About 15.0 cm.
   Diameter.—About 1.0 cm.
   Internode length.—About 1.0 cm to 2.0 cm.
   Aspect.—About 30° from vertical.
   Strength.—Strong.
   Texture.—Smooth.
   Color.—RHS 143C.
Foliage description:
   Arrangement.—Leaves opposite, three or five leaflets.
   Length.—Up to 15.0 cm.
   Width.—About 13.0 cm.
   Shape.—Ovate.
   Apex.—Acute to obtuse.
   Base.—Acute to obtuse.
   Margin.—Moderately serrated.
   Venation pattern.—Pinnate.
   Venation color.—RHS 139B.
   Texture.—Both upper and lower surfaces: Pilose.
   Color.—Both immature and mature: Upper surface: RHS 139A Lower surface: RHS N138C.
Petioles:
   Length.—2.0 cm until the first leaflet pair, 3 to 5 cm till the second leaflet pair.
   Diameter.—About 0.7 cm.
   Texture.—Upper and lower surfaces: Smooth.
   Color.—Upper and lower surfaces: RHS 138B.
Inflorescence description:
   Appearance and arrangement.—Daisy-eyed double inflorescences above the foliar plant.
   Fragrance.—Present.
   Time to flower.—Winter: About 3 months after planting Summer: About 2 months after planting.
   Post-production longevity.—Flower continuously during summer and autumn.
Inflorescence bud:
   Height.—About 1.0 cm.
   Diameter.—1.0 to 1.5 cm.
   Shape.—Oblate.
   Color.—RHS 144B and RHS 144A towards the base.
Inflorescence size:
   Diameter.—About 10.0 cm.
   Depth (height).—About 5.0 cm.
   Disc diameter.—About 2.0 cm.
   Receptacle height.—1.5 to 2.0 cm.
   Receptacle diameter.—4.0 cm.
   Receptacle color.—RHS 141B.
Ray florets:
   Quantity per inflorescence.—About 50 arranged in 3 to 4 whorls.
   Length.—About 3.5 cm.
   Width.—About 2.5 cm.
   Shape.—Elliptical.
   Apex.—Acute to obtuse.
   Base.—Acute.
   Margin.—Entire.
   Aspect.—Semi-upright.
   Texture.—Upper to lower surface: Smooth.
   Color.—Immature: Upper surface: RHS 9A and RHS 45B Lower surface: RHS 9B and RHS 45C Mature: Upper surface: RHS 9A and RHS 42A basal Lower surface: RHS 9B and RHS 42B basal.
Disc florets:
   Quantity per inflorescence.—About 60.
   Length.—About 1.0 cm.
   Diameter.—0.1 cm.
   Shape.—Tubular.
   Texture.—Inner and outer surfaces: Smooth.
   Color.—Immature and mature: Inner and outer surfaces: RHS 12B Mature: Inner and outer surfaces: RHS 12B.
Phyllaries:
   Quantity per inflorescence.—9.
   Length.—2.0 cm.
   Width.—1.0 cm.
   Shape.—Oblong.
   Apex.—Obtuse.
   Base.—Truncase.
   Margin.—Absent.
   Texture.—Upper and lower surfaces: Smooth.
   Color.—Upper and lower surfaces: RHS 144B basal, RHS 144D apex.
Peduncles:
   Length, terminal peduncle.—9.0 cm.
   Length, axillary peduncle.—6.0 cm.
   Diameter.—0.4 cm.
   Aspect.—Semi-upright.
   Strength.—Strong.
   Texture.—Smooth.
   Color.—RHS 143B.
Reproductive organs:
   Androecium.—Only full developed on disc florets Quantity per floret: 4 Filament length: 0.3 cm Filament color: RHS 3B Anther shape: Lanceolate Anther length: 0.5 cm Anther color: RHS 15A Pollen amount: Abundant Pollen color: RHS 21A.
   Gynoecium.—Quantity per floret: 1 Pistil length: 1.5 cm Style length: 0.9 cm Style color: RHS 1D Stigma shape: Lanceolate pinnate Stigma color: RHS 13A Ovary color: RHS 154C.
   Fruit and seed set.—No fruit and seed set observed.
Disease and insect/pest resistance: No special observations were made.

### COMPARISON WITH KNOWN VARIETY

‘KLEDH13033’ is most similar to the commercial Dahlia plant named ‘KLEDH11031’ commercially known as ‘Yogi’ (U.S. application Ser. No.13/573,047). Differences between the two varieties are described in Table 1:

TABLE 1
Comparison with Known Variety

| | Variety | Commercial line |
|---|---|---|
| Trait | ‘KLEDH13033’ | ‘KLEDH11031’ |
| Ray florets color | Yellow with a red eye | Pink with purple/red eye |

We claim:

1. A new and distinct variety of Dahlia plant named ‘KLEDH13033’ as shown and described herein.
